(19) United States
(12) Patent Application Publication
(10) Pub. No.: US 2008/0188717 A1
(43) Pub. Date: Aug. 7, 2008

(54) Double-Slope Prism Module for Laryngoscope

(76) Inventors: Tien-Sheng Chen, Taipei City (TW); Hui-Bih Yuan, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/766,501

(22) Filed: Jun. 21, 2007

(30) Foreign Application Priority Data
Feb. 7, 2007 (TW) 096202389

(51) Int. Cl. A61B 1/267; G02B 5/04

(52) U.S. Cl. 600/199; 359/837; 600/185

(57) ABSTRACT

A prism module for a laryngoscope is disclosed. Said prism module mainly comprises a substantially cylinder-shaped first main part extending along a first axis; and a first refractive surface and a second refractive surface located at two ends of the first main part, respectively. Said first main part extends along a first axis and defines a first angle and a second angle with the first refractive surface and the second refractive surface respectively, wherein the first angle and the second angle are both less than 90 degrees.

### BACKGROUND OF THE INVENTION

1. Field of the Invention

The present invention relates to a prism module for a laryngoscope. More specifically, this invention relates to a double-slope prism module for enhancing the viewing capability of a laryngoscope.

2. Description of the Related Art

Endotracheal intubation is a critical means for maintaining the breathing function of a patient under general anaesthesia. In most cases, to prevent the occurrence of hypoxia, anaesthetists have to complete the intubation by inserting an endotracheal tube into patients' trachea in a very short period of time to provide oxygen thereinto promptly. Therefore, it is extremely important for anaesthetists to perform the intubation efficiently.

Practically, to intabate quickly, most anaesthetists take advantage of a laryngoscope as a means to observe the condition of a patient's upper airway. Please refer to FIG. 1. An early laryngoscope 10P is mainly consisted of a handgrip 20, a blade 30, and an illumination unit 40P; in addition, a prism module 50P having a slope may be secured to the blade 30 to refract the observation angle, helping the anaesthetists observe the trachea more clearly. Refer now to FIG. 2. Before putting the blade 30 into a patient's mouth, an anaesthetist may have the patient lay face up and raise the patient's jaw first; after that, he/she may press the tongue base down with the blade 30 by holding the handgrip 20 so as to raise the epiglottis cartilage. In the meantime, light may be refracted thereby due to the installation of the prism module 50P, largely increasing the chance that the anaesthetist can see the trachea. However, because the anatomy of different patients sometimes varies a lot, the early prism module 50P may not work equally well in different situations. For example, in the case where the patient's tongue is too thick, as shown in FIG. 2, the anaesthetist may probably see the tonsil or esophagus only; therefore, the intubation may be impeded by the anaesthetist's failure to see where the trachea is. Thus, there is a need for a novel laryngoscope to provide a better view in different cases.

### SUMMARY OF THE INVENTION

It is therefore an object of the present invention to provide a double-slope prism module, which, when secured to a laryngoscope, may help users observe the trachea.

It is another object of the present invention to provide a laryngoscope secured by a double-slope prism module; said laryngoscope may enable users to view farther into the upper airway.

Certain of the foregoing and related objects are readily obtained according to the present invention in a prism module which mainly comprises: a substantially cylinder-shaped first main part extending along a first axis; and a first refractive surface and a second refractive surface located at two ends of the first main part, respectively, wherein the first axis defines a first angle and a second angle with the first refractive surface and the second refractive surface respectively, and the first angle and the second angle are both less than 90 degrees.

In this invention, light may be refracted twice by the double-slope design. Accordingly, when compared with the conventional single-slope prism module, which may only be secured to the end of the blade in proximity to the handgrip due to the limitation of observation angle, the prism module of the present invention may be secured to the position of the blade which is farther from the handgrip. As a result, users are capable of viewing the images of the deeper position in the upper airway and the opening of the trachea. Meanwhile, the prism module may further comprise a second main part as well as a connection surface and an observation surface located at two ends of the second main part, respectively. The second main part is cylindrical in shape and extends along a second axis; the connection surface is connected to the second refractive surface, and the observation surface is substantially perpendicular to the second axis, wherein the observation surface may be a convex so as to enlarge the image therethrough.

Furthermore, the present invention also provides a laryngoscope, mainly comprising: a handgrip; a blade connected to the handgrip; and a prism module secured to the blade. Said prism module mainly comprises: a substantially cylinder-shaped first main part extending along a first axis; and a first refractive surface and a second refractive surface located at two ends of the first main part, respectively, wherein the first axis defines a first angle and a second angle with the first refractive surface and the second refractive surface respectively, and the first angle and the second angle are both less than 90 degrees It is appreciated that in all of the above aspects of the invention, the prism module may be secured to the blade by means of buckling; however, it may also be fabricated on the blade integrally in a one-piece manner.

In order to cover a larger visual field, the first refractive surface of the prism module may be fabricated into a concave with a specific curvature; on the other hand, when users want to have an enlarged view of the object to be observed, the second refractive surface of the prism module may be fabricated into a convex with a specific curvature.

In addition, a light guide tube which is conventionally used as the illumination unit may be replaced by a smaller LED light bulb so as to leave more space on the blade for the accommodation of the prism module.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustrations only, and not as a definition of the invention.

In the drawings, wherein similar reference numerals denote similar elements throughout the several views:

FIG. 1 is a perspective diagram of a conventional laryngoscope.

FIG. 2 is a schematic diagram for a conventional laryngoscope under use.

FIG. 3 is an illustrative diagram of the first embodiment of the prism module of the present invention.

FIG. 4 is an illustrative diagram of the second embodiment of the prism module of the present invention.

FIG. 5 is an illustrative diagram of the third embodiment of the prism module of the present invention.

FIG. 6 is an illustrative diagram of the fourth embodiment of the prism module of the present invention.

FIG. 7 is a perspective diagram of an embodiment of the laryngoscope of the present invention.

FIG. 8 is a perspective diagram of another embodiment of the laryngoscope of the present invention.

FIG. 9 is a schematic diagram for the laryngoscope of the present invention under use.

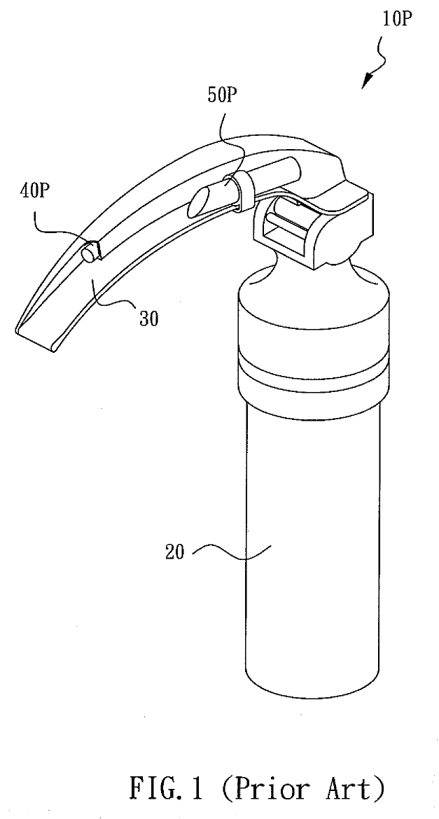

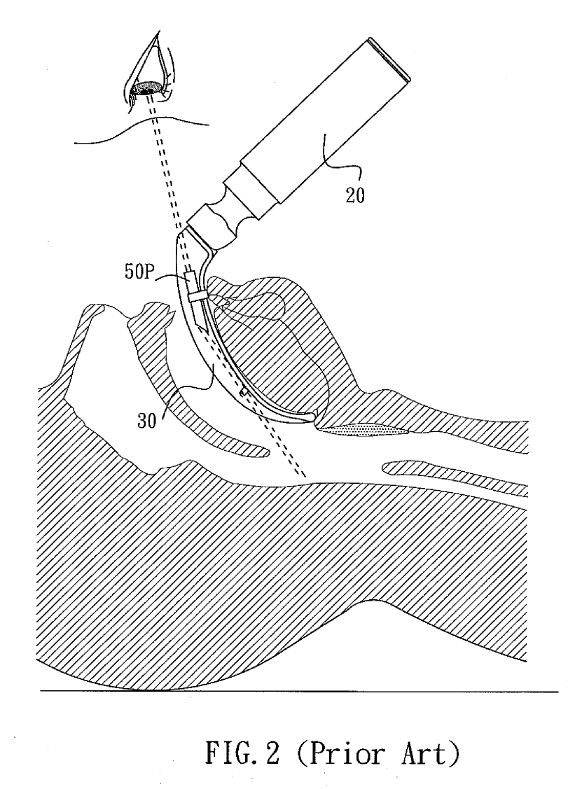

### DETAILED DESCRIPTION OF THE PREFERRED EMBODIMENT

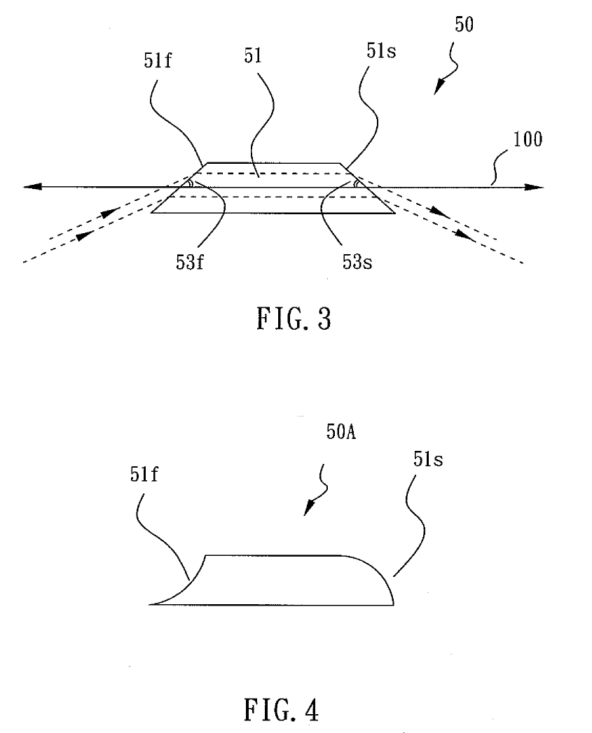

Please refer to FIG. 3 for an illustrative diagram of the first embodiment of the prism module 50 of the present invention. As shown, the prism module 50 substantially comprises a substantially cylinder-shaped first main part 51 extending along a first axis 100 and a first refractive surface 51f and a second refractive surface 51s located at two ends of the first main part 51, respectively. The first axis 100 defines a first angle 53f and a second angle 53s with the first refractive surface 51f and the second refractive surface 51s respectively, and the first angle 53f and the second angle 53s are both less than 90 degrees

As shown in FIG. 3 when incident light passes through the first refractive surface 51f, a first refraction may happen and the direction of the incident light may be deflected; in contrast, when the light is to leave from the second refractive surface 51s, a second refraction may happen and the direction of the light may be deflected again. It is the double refraction that enables users to observe the image of the deeper position in the upper airway and to position the location of the trachea as quickly as possible.

Refer now to FIG. 4 for an illustrative diagram of the second embodiment of the prism module 50A of the present invention. In this embodiment, the first refractive surface 51f and the second refractive surface 51s are fabricated into a concave and a convex respectively to increase the visual field. By the concave design, the visual field accommodated on the first refractive surface 51f may be increased so that the users may gain a better understanding of the situation in the patient's upper airway. In addition, to avoid an overly miniature image being formed on the second refractive surface 51s by a exceedingly large visual field, the second refractive surface 51s may be fabricated into a convex so as to enlarge the image properly.

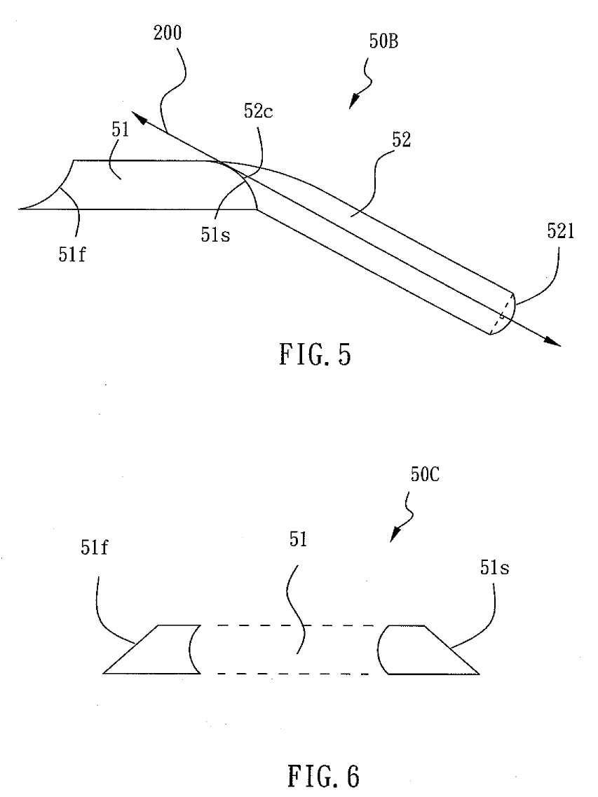

Moreover, in order to allow the users to observe from a more comfortable angle, the prism module of the invention may also adapt a dual-prism structure. Refer now to FIG. 5 for an illustrative diagram of the third embodiment of the prism module 50B of the present invention. In addition to the first main part 51, the first refractive surface 51f and the second refractive surface 51s disclosed in the second embodiment, the prism module SOB in this embodiment may further comprise a substantially cylinder-shaped second main part 52 extending along a second axis 200, as well as a connection surface 52c and an observation surface 521 located at two ends of the second main part 52, respectively. The connection surface 52c is connected to the second refractive surface 51s, and the observation surface 521 is substantially perpendicular to the second axis 200. The observation surface 521 may be fabricated into a convex so as to magnify the image thereon It should be noted that, in addition to the conventional prism structure, the main part(s) of the prism module may be realized by a light pipe as well. Refer to FIG. 6 for an illustrative diagram of the fourth embodiment of the prism module SOC of the present invention. In this embodiment, the first main part 51 is a light-impermeable light pipe, and the first refractive surface 51f and the second refractive surface 51s are the surfaces of two prisms; in addition, the two sides opposite to the first refractive surface 51f and the second refractive surface 51s on the two prisms may be fabricated into a concave and a convex, respectively. In this way, users are capable of observing the image with a desirable size and scope by the properly diverged and converged light.

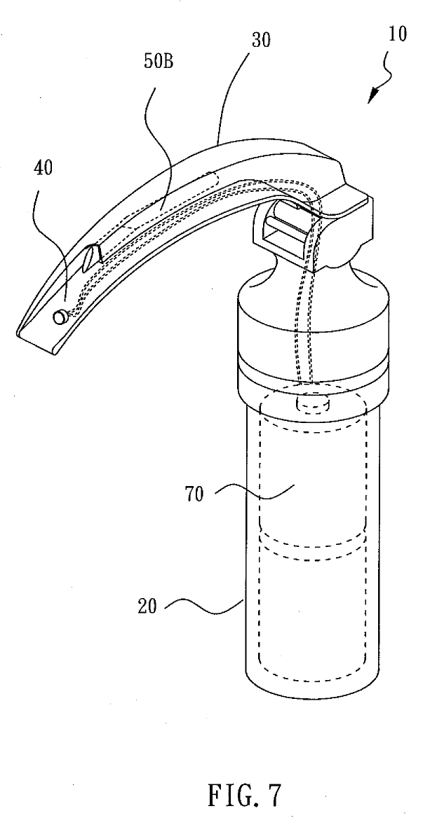

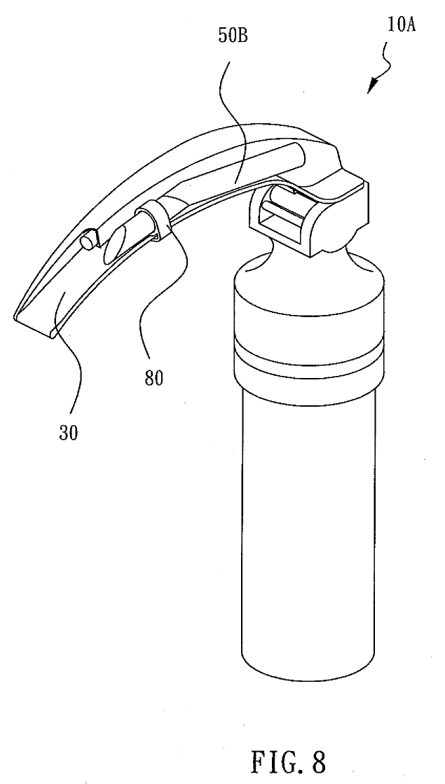

Please refer now to FIG. 7 for a perspective diagram of an embodiment of the laryngoscope 10 of the present invention. The laryngoscope 10 of the present invention mainly comprises a handgrip 20, a blade 30, and a prism module SOB, wherein the blade 30 is connected to the handgrip 20, and the prism module 50B may be formed on the blade 30 integrally in a one-piece manner. Undoubtedly, the prism module SOB may also be detachably secured on the blade 30. Also, refer to FIG. 8 for a perspective diagram of another embodiment of the laryngoscope 10A of the present invention. In this embodiment, the prism module 50B is secured to the blade 30 by a clip 80. Since the prism module 50B is movably secured, the users may adjust it to a preferable position as they wish. Different from the structure shown in FIG. 8, the prism module 50B illustrated in FIG. 7 is installed at the place where a traditional light guide pipe may occupy, and the illumination unit 40 is installed at a non-conventional place on the blade 30; because the illumination unit 40 in this embodiment is a smaller LED light bulb, the structure of this embodiment is more space-efficient than traditional ones, making the intubation of endotracheal tube easier. In addition, the illumination unit 40 is electrically connected to a power supply 70 (such as batteries) installed in the handgrip 20. By the double-slope design of the prism module SOB, the laryngoscope 10 of this invention may provide a better view than traditional ones; also, the prism module 50B may be secured at a position on the blade 30 which is farther from the handgrip 20 so as to enable the users to get a deeper view.

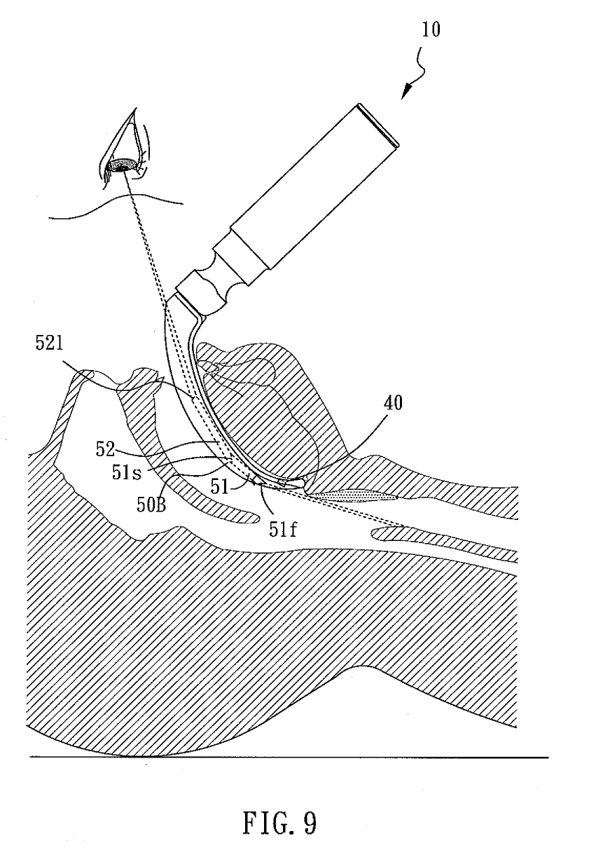

Last but not least, refer to FIG. 9 for a schematic diagram for the laryngoscope 10 of the present invention under use. As shown by the dash lines, the light reflected by the trachea may be refracted by the first refractive surface 51f first and proceed axially along the first main part 51 of the prism module 50B. When the light passes the second refractive surface 51s, it may be refracted again and directed into the second main part 52, along which it moves axially. When the light is emitted from the observation surface 521, which may be fabricated into a convex, the light may be properly magnified thereby before entering the users' eyes. Thus, the users may easily see the image therefrom and proceed with the intubation accordingly.

It will be understood that many other modifications can be made to the various disclosed embodiments without departing from the spirit and scope of the invention. For these reasons, the above description should not be construed as limiting the invention, but should be interpreted as merely exemplary of preferred embodiments.

What is claimed is:

1. A prism module to be secured to a laryngoscope, said prism module comprising:
a substantially cylinder-shaped first main part extending along a first axis; and
a first refractive surface and a second refractive surface located at two ends of the first main part, respectively,
wherein the first axis defines a first angle and a second angle with the first refractive surface and the second refractive surface respectively, and the first angle and the second angle are both less than 90 degrees.

2. The prism module as claimed in claim 1, wherein the first refractive surface is a concave.

3. The prism module as claimed in claim 1, wherein the second refractive surface is a convex.

4. The prism module as claimed in claim 1, wherein the first main part is a hollow light pipe.

5. The prism module as claimed in claim 1, further comprising:
a substantially cylinder-shaped second main part extending along a second axis; and
a connection surface and an observation surface located at two ends of the second main part, respectively,
wherein the connection surface is connected to the second refractive surface, and the observation surface is substantially perpendicular to the second axis.

6. The prism module as claimed in claim 5, wherein the observation surface is a convex.

7. The prism module as claimed in claim 5, wherein the second main part is a hollow light pipe.

8. A laryngoscope, comprising:
a handgrip;
a blade connected to the handgrip; and
a prism module secured to the blade, said prism module comprising:
a substantially cylinder-shaped first main part extending along a first axis; and
a first refractive surface and a second refractive surface located at two ends of the first main part, respectively,
wherein the first axis defines a first angle and a second angle with the first refractive surface and the second refractive surface respectively, and the first angle and the second angle are both less than 90 degrees.

9. The laryngoscope as claimed in claim 8, further comprising:
a power supply installed in the handgrip; and
an illumination unit connected electrically to the power supply.

10. The laryngoscope as claimed in claim 9, wherein the illumination unit is an LED light bulb.

11. The laryngoscope as claimed in claim 8, wherein the first refractive surface is a concave.

12. The laryngoscope as claimed in claim 8, wherein the second refractive surface is a convex.

13. The laryngoscope as claimed in claim 8, wherein the first main part is a hollow light pipe.

14. The laryngoscope as claimed in claim 8, wherein said prism module further comprises:
a substantially cylinder-shaped second main part extending along a second axis; and
a connection surface and an observation surface located at two ends of the second main part, respectively,
wherein the connection surface is connected to the second refractive surface, and the observation surface is substantially perpendicular to the second axis.

15. The laryngoscope as claimed in claim 14, wherein the observation surface is a convex.

16. The laryngoscope as claimed in claim 14, wherein the second main part is a hollow light pipe.
